(19) United States
(12) Patent Application Publication
(10) Pub. No.: US 2014/0276684 A1
(43) Pub. Date: Sep. 18, 2014

(54) ATHERECTOMY METHODS USING COREGISTERED SETS OF DATA

(71) Applicant: VOLCANO CORPORATION, San Diego, CA (US)

(72) Inventors: Scott Huennekens, San Diego, CA (US); Neil Hattangadi, San Diego, CA (US)

(73) Assignee: VOLCANO CORPORATION, San Diego, CA (US)

(21) Appl. No.: 14/204,107

(22) Filed: Mar. 11, 2014

Related U.S. Application Data
(60) Provisional application No. 61/792,230, filed on Mar. 15, 2013.

(51) Int. Cl. A61B 17/3207

(52) U.S. Cl. CPC A61B 17/3207
USPC 606/7; 606/159

(57) ABSTRACT

The present invention generally relates to methods for removing plaque from a vessel. The method can involve obtaining a first image of a blood vessel using a first imaging modality, obtaining a second image of the blood vessel using a second imaging modality, and coregistering the first and second images, thereby generating a coregistered data set. The method can further involve inserting an atherectomy catheter into the blood vessel and removing plaque from the vessel with the atherectomy catheter based on plaque identified in the coregistered set of data.

### RELATED APPLICATION

The present invention claims the benefit of and priority to U.S. Provisional No. 61/792,230, filed Mar. 15, 2013, which is incorporated by reference in its entirety.

### FIELD OF THE INVENTION

The present invention generally relates to atherectomy procedures performed using coregistered sets of data.

### BACKGROUND

Cardiovascular disease frequently arises from the accumulation of atheromatous deposits on the inner walls of vessel lumen, particularly the arterial lumen of the coronary and other vasculature, resulting in a condition known as atherosclerosis. These deposits can have widely varying properties, with some deposits being relatively soft and others being fibrous and/or calcified. In the latter case, the deposits are frequently referred to as plaque. These deposits can restrict blood flow, which in severe cases can lead to myocardial infarction.

Traditional methods of dealing with occluded arteries include bypass surgery. Bypass surgery typically involves obtaining vascular tissue from another part of the patient's body, such as the leg, and using the tissue to construct a shunt around the obstructed vessel. Bypass surgery is considered to be a relatively complex and risky procedure.

One alternative to bypass surgery is an atherectomy. An atherectomy involves physically breaking up the material occluding the vessel. In this procedure, an atherectomy catheter is inserted into the femoral artery through a small hole made therein and used to clear obstructions in the occluded area by grinding, aspirating, or cutting away atherosclerotic plaque build-up. Although an atherectomy is considered to be less invasive and therefore, poses less risk than a bypass, the procedure is not without its challenges. A primary concern is that the atherectomy catheter will cut beyond the plaque and damage healthy tissue. This is especially dangerous as the healing of the wounded vessel may lead to smooth muscle cell proliferation or a general healing response that can again occlude the vessel, in a phenomenon known as restenosis.

### SUMMARY

The present invention provides a method for performing an atherectomy utilizing multimodal coregistered sets of data to facilitate the removal of plaque and minimize the incidence of cutting into healthy tissue. The invention involves the coregistration of, for example, an intravascular ultrasound (IVUS) image with an angiography image. After the IVUS catheter used to obtain the IVUS image is withdrawn from the body, the method can further involve inserting an atherectomy catheter into the vessel. Plaque is identified based on the coregistered IVUS/angiography image, and the atherectomy catheter is then used to excise the identified plaque. In preferred aspects of the invention, the position and/or orientation of the atherectomy catheter is also coregistered to the IVUS/angiogram data set.

As encompassed by the invention, the coregistered sets of data are used to identify and distinguish plaque from healthy tissue. The coregistered set of data can include data from an internal imaging modality, including IVUS, and an external imaging modality, such as x-ray angiography. Further modalities suitable for use with the invention, include without limitation, optical coherence tomography (OCT), external ultrasound, computed tomography angiography (CTA), and magnetic resonance angiography (MRA). The invention may also incorporate the use of functional data, such as pressure or flow within a vessel. Once the coregistered data set has been obtained, the data is then used to by the operator to determine where plaque is to be removed within the vessel, such that the operator can cut away at the plaque without fear of cutting healthy tissue.

To further facilitate the identification of plaque, methods of the invention can also incorporate the use of virtual histology intravascular ultrasound (VH-IVUS) to help distinguish between healthy tissue and plaque. VH-IVUS utilizes the IVUS signal to create color-coded maps that overlay traditional gray-scale IVUS images, for purposes of distinguishing between areas of different histological structure (i.e., between healthy tissue and plaque or between various stages of plaque). Accordingly, methods of the invention can be used not only to differentiate plaque from the vessel wall, but also to characterize the type of plaque so that the appropriate atherectomy procedure can be selected.

As noted above, the position of the inserted atherectomy catheter may be coregistered to the IVUS/angiogram data set. This facilitates tracking the location of the device through the vessel lumen. To aid in this process, the catheter may include one or more markers that are visible to the external imaging modality. For example, the catheter may feature radiopaque markers detectable by x-ray angiography. The configuration of markers on the device also allows the determination of its orientation within the vessel. For example, the markers can be staggered, which enable the rotational orientation of the device to be determined.

The invention also provides for devices for use in practicing the above methods. In one aspect, the invention provides an atherectomy catheter. Atherectomy catheters in accordance with the invention can include the coregisterable markers discussed above, as well as one or more imaging sensors. The imaging sensor may include an IVUS transducer. As the provided catheter cuts through the plaque, the original IVUS-coregistered image can be refreshed with images obtained from the IVUS transducer, allowing the physician to monitor the progress of the procedure.

In light of the above, methods and devices of the invention facilitate the removal of plaque from an occluded vessel while mitigating the risk of damaging healthy tissue. Accordingly, the atherectomy procedure is more efficient and safer using the provided methods and devices.

### BRIEF DESCRIPTION OF THE DRAWINGS

FIG. 1 is a schematic illustration of a system for implementing intravascular image co-registration.

FIG. 2 depicts an illustrative angiogram image.

FIG. 3 depicts an illustrative fluoroscopic image of a radiopaque marker mounted upon a catheter.

FIG. 4 depicts an illustrative enhanced radiological image alongside a cross-sectional IVUS image.

FIG. 5 is a flow chart, delineating at least of some of the steps which are used to automatically generate a road map, in accordance with some applications of the present invention.

FIG. 6 illustrates phased-array imaging catheter suitable for use in methods of the invention.

FIG. 7 illustrates a rotational imaging catheter suitable for use in methods of the invention.

FIG. 8 illustrates a guidewire with functional sensors for use in methods of the invention.

FIG. 9 illustrates a system for use in methods of the invention.

FIGS. 10-13 depict various embodiments of a distal end of an atherectomy tool suitable for use in methods of the invention.

### DETAILED DESCRIPTION

The present invention provides methods for performing atherectomies. The invention utilizes coregistered sets of data from different modalities to facilitate the removal of plaque and minimize the incidence of cutting into healthy tissue. In certain aspects, the invention encompasses obtaining a first set of image data corresponding to a blood vessel using a first imaging modality, obtaining a second set of image data corresponding to the blood vessel using a second imaging modality, and coregistering the first and second sets of image data, thereby generating a coregistered set of image data. The invention further encompasses identifying vessel plaque in the coregistered set of image data, inserting a plaque-removal device into the blood vessel, and removing plaque within the vessel using the device based on plaque identified in the coregistered set of image data.

The alignment of positional data from multiple imaging modalities is typically referred to as co-registration. Co-registration generally refers to any method of re-aligning images, and in particular aligning or overlaying images from different modalities. Co-registration is often used to overlay structural and functional images as well as link functional scans to anatomical scans. The co-registration of images and positional data from multiple imaging modalities is known in the art. Details regarding image co-registration can be found in, for example, in U.S. Pat. Nos. 7,930,104; and 8,298,147; and U.S. patent application Ser. No. 13/388,932, each of which is incorporated herein by reference.

An exemplary method of co-registration is now described which uses x-ray fluoroscopy and intravascular ultrasound to obtain a co-registered intravascular data set. The invention, however, encompasses any and all imaging modalities, including without limitation, intravascular ultrasound (IVUS), optical coherence tomography (OCT), external ultrasound, x-ray angiography, Computerized Tomography (CT) angiography, and Magnetic Resonance (MR) angiography. Such modalities can be used instead of x-ray fluoroscopy and intravascular ultrasound and also in addition to such modalities. Any number of modalities is useful for coregistration. Furthermore, modalities suitable for coregistration include functional measurement parameters, including vessel flow, vessel pressure, FFR, iFR, CFR, etc.

Turning initially to FIG. 1, an exemplary system is schematically depicted for carrying out the present invention in the form of co-registration of angiogram/fluoroscopy and intravascular ultrasound images. The radiological and ultrasound image data acquisition sub-systems are generally well known in the art. With regard to the radiological image data, a patient 10 is positioned upon an angiographic table 12. The angiographic table 12 is arranged to provide sufficient space for the positioning of an angiography/fluoroscopy unit c-arm 14 in an operative position in relation to the patient 10 on the table 12. Radiological image data acquired by the angiography/fluoroscopy c-arm 14 passes to an angiography/fluoroscopy processor 18 via transmission cable 16. The angiography/fluoroscopy processor 18 converts the received radiological image data received via the cable 16 into angiographic/fluoroscopic image data. The angiographic/fluoroscopic (“radiological”) image data is initially stored within the processor 18.

With regard to portions of the system associated with acquiring ultrasound image data, an imaging catheter 20, and in particular an IVUS catheter, is inserted within the patient 10 so that its distal end, including a diagnostic probe 22 (in particular an IVUS probe), is in the vicinity of a desired imaging location of a blood vessel. While not specifically identified in FIG. 1, a radiopaque material located near the probe 22 provides indicia of a current location of the probe 22 in a radiological image. By way of example, the diagnostic probe 22 generates ultrasound waves, receives ultrasound echoes representative of a region proximate the diagnostic probe 22, and converts the ultrasound echoes to corresponding electrical signals. The corresponding electrical signals are transmitted along the length of the imaging catheter 20 to a proximal connector 24. IVUS versions of the probe 22 come in a variety of configurations including single and multiple transducer element arrangements. In the case of multiple transducer element arrangements, an array of transducers is potentially arranged: linearly along a lengthwise axis of the imaging catheter 20, curvilinearly about the lengthwise axis of the catheter 20, circumferentially around the lengthwise axis, etc.

The proximal connector 24 of the catheter 20 is communicatively coupled to a catheter image processor 26. The catheter image processor 26 converts the signals received via the proximal connector 24 into, for example, cross-sectional images of vessel segments. Additionally, the catheter image processor 26 generates longitudinal cross-sectional images corresponding to slices of a blood vessel taken along the blood vessel's length. The IVUS image data rendered by the catheter image processor 26 is initially stored within the processor 26.

The type of diagnostic imaging data acquired by the diagnostic probe 22 and processed by the catheter image processor 26 varies in accordance with alternative embodiments of the invention. In accordance with a particular alternative embodiment, the diagnostic probe 22 is equipped with one or more sensors (e.g., Doppler and/or pressure) for providing hemodynamic information (e.g., blood flow velocity and pressure)—also referred to as functional flow measurements. In such alternative embodiments functional flow measurements are processed by the catheter image processor 26. It is thus noted that the term “image” is intended to be broadly interpreted to encompass a variety of ways of representing vascular information including blood pressure, blood flow velocity/volume, blood vessel cross-sectional composition, shear stress throughout the blood, shear stress at the blood/blood vessel wall interface, etc. In the case of acquiring hemodynamic data for particular portions of a blood vessel, effective diagnosis relies upon the ability to visualize a current location of the diagnostic probe 22 within a vasculature while simultaneously observing functional flow metrics indicative of cardiovascular disease. Co-registration of hemodynamic and radiological images facilitates precise treatment of diseased vessels. Alternatively, instead of catheter mounted sensors, the sensors can be mounted on a guidewire, for example a guidewire with a diameter of 0.018″ or less. Thus, in accordance with embodiments of the present invention, not only are a variety of probe types used, but also a variety of flexible elongate members to which such probes are mounted at a distal end (e.g., catheter, guidewire, etc.).

A co-registration processor 30 receives IVUS image data from the catheter image processor 26 via line 32 and radiological image data from the radiological image processor 18 via line 34. Alternatively, the communications between the sensors and the processors are carried out via wireless media. The co-registration processor 30 renders a co-registration image including both radiological and IVUS image frames derived from the received image data. In accordance with an embodiment of the present invention, indicia (e.g., a radiopaque marker artifact) are provided on the radiological images of a location corresponding to simultaneously displayed IVUS image data. The co-registration processor 30 initially buffers angiogram image data received via line 34 from the radiological image processor 18 in a first portion 36 of image data memory 40. Thereafter, during the course of a catheterization procedure IVUS and radiopaque marker image data received via lines 32 and 34 is stored within a second portion 38 and a third portion 42, respectively, of the image data memory 40. The individually rendered frames of stored image data are appropriately tagged (e.g., time stamp, sequence number, etc.) to correlate IVUS image frames and corresponding radiological (radiopaque marker) image data frames. In an embodiment wherein hemodynamic data is acquired rather than IVUS data, the hemodynamic data is stored within the second portion 38.

In addition, additional markers can be placed on the surface of the patient or within the vicinity of the patient within the field of view of the angiogram/fluoroscope imaging device. The locations of these markers are then used to position the radiopaque marker artifact upon the angiographic image in an accurate location.

The co-registration processor 30 renders a co-registration image from the data previously stored within the first portion 36, second portion 38 and third portion 42 of the image data memory 40. By way of example, a particular IVUS image frame/slice is selected from the second portion 38. The co-registration processor 30 identifies fluoroscopic image data within the third portion 42 corresponding to the selected IVUS image data from the second portion 38. Thereafter, the co-registration processor 30 superimposes the fluoroscopic image data from the third portion 42 upon the angiogram image frame retrieved from the first portion 36. Thereafter, the co-registered radiological and IVUS image frames are simultaneously displayed, along-side one another, upon a graphical display device 50. The co-registered image data frames driving the display device 50 are also stored upon a long-term storage device 60 for later review in a session separate from a procedure that acquired the radiological and IVUS image data stored in the image data memory 40.

While not shown in FIG. 1, a pullback device is incorporated that draws the catheter 20 from the patient at a controlled/measured manner. Such devices are well known in the art. Incorporation of such devices facilitates calculating a current position of the probe 22 within a field of view at points in time when fluoroscopy is not active.

Turning to FIG. 2, the angiography/fluoroscopy processor 18 captures an angiographic “roadmap” image 200 in a desired projection (patient/vessel orientation) and magnification. By way of example, the image 200 is initially captured by an angiography procedure performed prior to tracking the IVUS catheter to the region of interest within a patient's vasculature. Performing the angiography procedure without the catheter 20 in the vessel provides maximal contrast flow, better vessel filling and therefore a better overall angiogram image. Thus, side branches such as side branch 210 and other vasculature landmarks can be displayed and seen clearly on the radiological image portion of a co-registered image displayed upon the graphical display device 50.

Turning to FIG. 3, the catheter 20 is tracked to its starting position (e.g., a position where an IVUS pullback procedure begins). Typically the catheter 20 is tracked over a previously advanced guidewire (not shown). Thereafter, a fluoroscopic image is obtained. In the image, the catheter radiopaque marker 300 is visualized, but the vessel lumen is not, due to the absence of contrast flow. However, a set of locating markers present in both the angiogram and fluoroscopy images enable proper positioning (superimposing) of the marker image within the previously obtained angiogram image. Other ways of properly positioning the radiopaque marker image within the field of view of the angiogram image will be known to those skilled in the art in view of the teachings herein. Furthermore, the marker artifact can be automatically adjusted (both size and position) on the superimposed image frames to correspond to the approximate position of the transducers. The result of overlaying/superimposing the radiopaque marker artifact upon the angiogram image is depicted, by way of example in an exemplary co-registration image depicted in FIG. 4.

Turning to FIG. 4 the exemplary co-registration display 401 (including the correlated radiological and IVUS images) depicts a selected cross-sectional IVUS image 400 of a vessel. A radiological image 410 is simultaneously displayed along-side the IVUS image 400 on the display 50. The radiological image 410 includes a marker artifact 420, generated from radiological image data rendered by a fluoroscope image frame, superimposed on an angiogram background rendered from the first portion 36 of the memory 40. The fluoroscope image frame corresponds to the current location of the diagnostic probe 22 within a vessel under observation. Precise matching of the field of view represented in both the angiogram and fluoroscope images (i.e., precise projection and magnification of the two images) allows identification of the current position of the IVUS probe corresponding to the displayed IVUS image 400 in the right pane of the co-registered images displayed in FIG. 4. As noted above, further detail on image co-registration may be found in U.S. Pat. No. 7,930,014, incorporated herein by reference.

Although the operator will be able to identify vessel plaque from the coregistered data set discussed above and thus proceed with the atherectomy based on such information, methods of the invention may also encompass the use of virtual histology to further facilitate the identification of plaque. Intravascular ultrasound (“IVUS”) combined with virtual histology (“VH”) has been particularly successful in recognizing the morphology of atherosclerotic plaque in vivo (i.e., the location and composition of plaque in the patient's body). Virtual histology technology can be incorporated into the imaging systems used with the invention (an IVUS imaging system, for example) to help the physician recognize and identify the morphology of tissue, particularly plaque associated with a lesion, in vivo (i.e., the location and composition of plaque in the patient's body). The following systems for detecting and characterizing plaque using IVUS with VH are disclosed in U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

In one embodiment of VH technology, an ultrasonic device is used to acquire RF backscattered data (i.e., IVUS data) from a blood vessel. The IVUS data is then transmitted to a computing device and used to create an IVUS image. The blood vessel is then cross-sectioned and used to identify its tissue type and to create a corresponding image (i.e., histology image). A region of interest (ROI), preferably corresponding to the identified tissue type, is then identified on the histology image. The computing device, or more particularly, a characterization application operating thereon, is then adapted to identify a corresponding region on the IVUS image. To accurately match the ROI, however, it may be necessary to warp or morph the histology image to substantially fit the contour of the IVUS image. After the corresponding region is identified, the IVUS data that corresponds to this region is identified. Signal processing is then performed and at least one parameter is identified. The identified parameter and the tissue type (e.g., characterization data) are stored in a database. In another embodiment of the present invention, the characterization application is adapted to receive IVUS data, determine parameters related thereto (either directly or indirectly), and use the parameters stored in the database to identify a tissue type or a characterization thereof.

Methods of the invention may also encompass the generation of a vascular road map, according to the following general process. Roadmaps may be generated using this process to provide an enhanced image relative to a conventional angiogram. It is contemplated that these enhanced images, such as an enhanced angiogram can be coregistered with another modality, such as IVUS or OCT using the methods described above. The processes described herein also facilitate the tracking of a medical device, such as an atherectomy catheter through the vessel. Although, the general process is provided here, further detail can be found in U.S. Patent Application Pub. 2010/0161023 to Cohen et al, incorporated herein by reference.

Reference is now made to FIG. 5, which is a flow chart delineating at least some of the steps of which are used to automatically generate a road map, in accordance with some applications of the present invention. The automatic generation of a road map is described with reference to coronary angiography, by way of example. The scope of the present invention includes the automatic generation of a road map using a different imaging modality.

In Phase 1 of the automatic road map generation, a fluoroscopic image stream of the coronary arteries is acquired. Typically, during the acquisition of the image stream, a contrast agent is administered to the subject. Optionally, the image stream is gated, tracked, and/or stabilized by other means. For example, selected image frames corresponding to a given phase in the motion cycle of the heart may be identified by means of a physiological signal. For some applications, the physiological signal applied is the subject's ECG and the image frames are selected by means of gating to the ECG and/or by other means of gating as described in WO 08/107,905 to Iddan, which is incorporated herein by reference. It is noted that stabilization of the image stream is optional and, for some applications, a road map is automatically generated on a native (non-stabilized) fluoroscopic image stream.

For some applications, the ECG signal is received from an ECG monitor. Alternatively or additionally, the ECG signal is received from a Cardiac Rhythm Management (CRM) device such as a pacer, or a defibrillator. For some applications, a processor that performs the automatic generation of the road map, or a dedicated processor, identifies the selected phase of the ECG signal. (In general, in the present application, when references are made to the functionalities of a processor, the functionalities may be performed by a single processor, or by several processors, which act, effectively, like a single processor with several functionalities.) Alternatively, the selected phase (e.g., the R wave of the ECG signal) is identified by the ECG monitor. Further alternatively, the selected phase (e.g., the R wave of the ECG signal) is identified by the CRM device.

For some applications, image tracking is applied to the native image stream, with respect to a guiding catheter or with respect to a segment of the guiding catheter, as described in further detail hereinbelow. For example, the native image stream may be image tracked with respect to the distal tip of the guiding catheter, e.g., a curved portion of the guiding catheter. Alternatively or additional, image tracking is performed with respect to one or more radiopaque (or otherwise visible) markers or segments of a tool. For some applications, image tracking, or alternative techniques for stabilizing the image stream, is performed with respect to a virtual feature or region of image frames of the native image stream. Such virtual features are typically derived from a manipulation (such as an average, a weighted average, a translation, a rotation, and/or a scaling) of the location of one or more observable features of the image. For example, the virtual feature may be the average location of two radiopaque markers of a balloon or atherectomy catheter.

In Phase 2 of the automatic road map generation, a baseline fluoroscopic image frame is identified, typically automatically, the baseline image frame having been acquired prior to the contrast agent having been administered to the subject. (For some applications, the baseline frame is selected manually by the user.) For some applications, the baseline image frame is gated to a given phase of the subject's cardiac cycle (i.e., it selected based on its having been acquired at the given phase of the subject's cardiac cycle). Typically, the baseline image is an image frame that is generated immediately before the contrast agent was (or is about to be) administered to the subject (as described in further detail hereinbelow).

For some applications, the baseline image frame is used a reference image frame, to which to compare subsequent image frames, in order to determine when an angiographic sequence has commenced, as described hereinbelow. Alternatively or additionally, techniques such as the techniques described hereinbelow are used for determining the commencement or the end of an angiographic sequence, not by comparing image frames to the baseline image frame, but by detecting rapid changes in parameters of image frames of the image stream. For example, in order to determine when an angiographic sequence has commenced, a vesselness descriptor may be calculated for each image in the image stream. The vesselness descriptor is typically calculated in accordance with the techniques described hereinbelow. For example, the vesselness descriptor may be calculated by counting a number of possible centerline points of a vessel in each of the images that are located near to possible edge lines of the vessel. Commencement of an angiographic sequence is determined by detecting a rapid increase in the vesselness descriptor. The end of an angiographic sequence is determined by detecting a rapid decrease in the vesselness descriptor.

For some applications, the baseline image frame is analyzed such that the degree of “vesselness” (i.e., the extent to which a given pixel is likely to be an element of an image of a vessel) in applicable areas of the image frame is determined. For example, vesselness may be determined by means of a filter, such as the filter described in the article by Frangi (a “Frangi filter”), cited hereinabove, which is incorporated herein by reference, and/or by means of a filter that performs enhancement and/or detection and/or segmentation of curvilinear structures. For some applications, a filter is used that is similar to a Frangi filter, but that differs from a Frangi filter (“a Frangi-like filter”) (a) in that vesselness is a homogeneous function, and/or (b) in the multipliers employed for the normalization of scales.

In Phase 3 of the automatic road map generation, an identification or detection is typically provided that angiography has commenced or is about to commence. For example, commencement of the angiography may be detected by detecting the injection of contrast agent, and/or by detecting the activation of a special imaging mode such as cine. For some applications, several angiographic sequences are acquired and the commencement of each of the angiographic sequences is detected, in order to separate the angiographic sequences from one another. Typically, the angiographic sequences are separated from each other such that the most suitable image frame for generating a new road map is selected only from among the frames belonging to the most recent angiographic sequence.

For some applications, the identification that angiography has commenced, or is about to commence, is provided automatically by the apparatus for injecting the contrast agent. Alternatively or additionally, the identification that angiography has commenced, or is about to commence, is provided manually by the operator of the apparatus injecting the contrast agent. Further alternatively or additionally, the identification that angiography has commenced is provided automatically by identifying that in the acquired image frames there is an increased portion or count of vessel-like pixels. For example, such automatic identification may be provided by means of a filter that performs enhancement and/or detection and/or segmentation of curvilinear structures, a Frangi filter, and/or a Frangi-like filter. For some applications, the commencement of an angiographic sequence is detected by detecting the appearance of temporarily-appearing vessel-like features. Typically, the detection of temporarily-appearing vessel-like features indicates a new angiographic sequence.

For some applications, the identification that angiography has commenced is provided automatically by means of image processing, as described in WO 08/107,905 to Iddan, which is incorporated herein by reference. Suitable image processing techniques include the analysis of changes in the current image, and/or, specifically, changes in the image region at the distal end of the catheter from which the contrast agent enters the subject's vasculature (such as a guiding catheter in the case of coronary road mapping). For example, changes in the image may include a relatively abrupt change in the color and/or grayscale level (i.e., darkness) of a relatively large number and/or portion of image pixels, or the appearance of vessel-like features in the image, or any combination thereof. It is noted that by assessing a change in the darkness level to identify the time of injection of the contrast agent, the automatic road map generation processor may identify a darker area of the image or a lighter area of the image, depending on whether the contrast agent is represented as dark or light.

For some applications, the identification that angiography has commenced is performed by comparing a current image frame to the baseline image frame. Alternatively, the identification that angiography has commenced is performed not by comparing image frames to the baseline image frame, but by detecting rapid changes in parameters of image frames of the image stream. For some applications, the identification that angiography has commenced is accelerated by reducing the resolution of the image frames, and applying image processing techniques to the reduced-resolution image frames.

It is noted that whereas specifically assessing the region at the distal end of the catheter typically enhances signal to noise (because this region is most likely to show an abrupt change), the scope of the present invention includes assessing most or all of the acquired image data to identify the injection of the contrast agent.

In Phase 4 of the automatic road map generation, an identification or detection is typically provided that the acquisition of image frames in the presence of contrast agent has ended or subsided. That is to say, the contrast agent injected into the coronary arteries has dissipated (or mostly dissipated) such that it is generally no longer visible in the fluoroscopic images. For some applications, such identification is provided automatically by apparatus that injects the contrast agent, and/or is provided manually by the operator of the apparatus injecting the contrast agent. For some applications, such identification or detection is provided by identifying decreased vesselness, for example, by means of a filter that performs enhancement and/or detection and/or segmentation of curvilinear structures, a Frangi filter, and/or a Frangi-like filter. Alternatively or additionally, such identification or detection is provided automatically by image processing techniques similar to those described with reference to Phase 3 above. For some applications, and as an alternative to Phase 4, the end of a sequence of angiographic images is assumed after a certain period of time has elapsed since the commencement of the angiographic sequence. The period of time typically corresponds to the typical duration of an angiographic sequence.

In Phase 5 of the automatic generation of the road map, the angiographic image frames (also known as angiograms) corresponding to a given angiographic sequence are automatically analyzed, such that an angiogram is derived (e.g., selected) from the set of angiograms, based upon visibility of at least a portion of the blood vessels in the angiograms. For some applications, the angiogram with the greatest visibility of coronary arteries is selected, with such selection typically being automatic. The greatest visibility is typically determined based upon the greatest total number of arteries observed, the greatest number of image pixels attributed to an artery, and/or the greatest image contrast in the appearance of specific arteries. Such an angiogram with the greatest visibility of coronary arteries is typically the most suitable for serving as the basis for the most informative road map in situations wherein the greatest amount of vasculature should be observed.

For some applications, an aggregated image of two or more angiograms is derived from the sequence of angiograms. For example, two or more angiograms that provide the greatest visibility of the coronary arteries are added to each other. Alternatively, a portion of a first angiogram that provides good visibility of a first portion of the coronary arteries is aggregated with a portion of a second angiogram that provides good visibility of a second portion of the coronary arteries.

For some applications, an angiogram having the greatest visibility of the coronary arteries is identified by means of vesselness of image pixels. Alternatively or additionally, such vesselness is determined by means of a filter, such as a filter that performs enhancement and/or detection and/or segmentation of curvilinear structures, a Frangi filter, and/or a Frangi-like filter. For some applications, the determination of vesselness of image pixels is made with reference to known anatomical structures, and/or with reference to known anatomy of the specific subject. For some applications, the determination of vesselness of image pixels is made while accounting for the specific viewing angle at which the images are generated.

For some applications, only angiograms belonging to the angiographic sequence that are gated to a given phase of the cardiac cycle are analyzed. An angiographic image frame is derived (e.g., selected) from the gated angiograms, based upon visibility of at least a portion of the blood vessels in the angiograms. For example, the gated angiogram with the greatest visibility of coronary arteries may be selected. For some applications, the given cardiac phase is an end-diastolic phase, at which certain coronary vessels are typically the most spread apart. For some applications, the end-diastolic phase is identified by means of image processing (and not, or not exclusively, by means of gating to the ECG signal). For example, an image in which distances between coronary vessels are largest may be identified, and/or a degree of vesselness within a region of interest may be analyzed. For some applications, an image frame in which motion of coronary blood vessels is at a minimum, as would typically be expected during end-diastole, is identified.

For some applications, limiting the derivation of the angiogram to only among angiograms gated to a specific cardiac phase is suitable when the operator's interest is focused on the specific phase. Typically, for such applications, the operator will designate the phase with respect to which the angiograms are gated via an input device (e.g., a keyboard, a mouse, a trackball, a touchscreen, a joystick, etc.). For some applications, only angiograms sampled at a defined time interval (e.g., every 100 ms, or between the 700th ms and 1000th ms of every second), and/or at a defined sequential interval (e.g., every fifth frame, or between the 10th and 15th of every 15 frames), are analyzed. For some applications, frames sampled within the time interval are gated, and/or frame(s) with the highest vesselness are identified from among frames sampled within the time interval.

In Phase 6 of the automatic road map generation, designated vessels in the selected angiogram(s) are enhanced, typically automatically. For some applications, low-contrast vessels that are typically less observable in the non-enhanced image, and/or narrow vessels that are typically less observable in the non-enhanced image, are detected and enhanced. For some applications, non-vascular structures whose spatial and/or temporal characteristics differ from those of vascular structures are identified, and the visibility of such structures is reduced. For example, such spatial characteristics may include dimensions, relative location, gray level, texture, edge smoothness, or any combination thereof, and such temporal characteristics may include relative motion, absolute motion, and/or a change over time of any of any of the aforementioned spatial characteristics. For some applications, the enhancement is performed by means of a filter that detects and/or segments curvilinear structures. Alternatively or additionally, the enhancement is performed by means of a Frangi-filter, such that vessels and their local orientation are automatically detected by analyzing eigenvalues and eigenvectors of the Hessian matrix of a smoothed image.

In Phase 7 of the automatic road map generation, the darkest lines, or the center lines, or any other characterizing or representative lines corresponding to paths of one or more designated blood vessels are determined, typically automatically. For some applications, the points comprising such lines are determined by means of their relatively high value of vesselness. Alternatively or additionally, the points comprising such lines are determined by the extent to which their gradient is orthogonal to the eigenvector of the Hessian matrix corresponding to the highest eigenvalue. For some applications, such determination is assisted by a voting function applied to points that are adjacent to those points that are eventually determined to constitute the center line itself.

In Phase 8 of the automatic road map generation, which is applicable in cases in which there are discontinuities within a center line (or any other characterizing or representative line) of a designated vessel, such discontinuities are bridged, typically automatically. For some applications, end points are identified automatically at both sides of a discontinuity. For some applications, bridging is performed across gaps between end points by means of a shortest-path algorithm, for example the shortest-path algorithm described in the article by Dijkstra, which is cited hereinabove, and which is incorporated herein by reference. For some applications, bridging is performed subsequent to the detection of edges (i.e., boundaries), corresponding to each already-determined segment of the center lines, i.e., subsequent to Phase 9 of the automatic road map generation, described hereinbelow.

For some applications, bridging is performed across gaps between end points by means of an algorithm that takes into account the directional vectors of the lines at both sides of the discontinuity. Alternatively or additionally, the bridging is performed with reference to known typical structures of the coronary tree. For example, bridging may be performed based upon what is typical at the corresponding section of a coronary tree.

For some applications, the bridging of gaps is performed with reference to known structures of the coronary tree of the particular subject who is being imaged. Typically, in such cases, gaps are bridged based upon what has been previously observed, by means of imaging a corresponding section of the subject's coronary tree. In accordance with respective applications, the imaging modality used to image the corresponding section of the subject's coronary tree is the same as the modality that is used to generate the angiograms, or is a different imaging modality (for example, pre-operative CT) from the imaging modality used to generate the angiograms (for example, fluoroscopy).

For some applications, the bridging of gaps is made while accounting for the specific viewing angle at which the images are generated.

In Phase 9 of the automatic road map generation, the boundaries (i.e., edges or edge lines) of one or more designated vessels are determined, typically automatically. For some applications, such boundaries are determined by means of region-based adaptive thresholding of the vesselness image. Alternatively or additionally, such boundaries are determined by means of a region-growing algorithm. Further alternatively or additionally, such boundaries are determined by means of an edge detector, and/or by means of a morphological operation. For some applications, such boundaries are determined by means of a watershed technique, which splits an image into areas, based on the topology of the image. Alternatively or additionally, such boundaries are determined by means of a live contour, and/or by means of matching filters.

In addition to the above methods for generating a vascular roadmap, methods of the invention also include tracking a virtual or actual medical tool through the road map. For example, based on the following description, one could track an atherectomy catheter through a roadmap (coregistered or otherwise) to direct the catheter to an area requiring plaque removal. In addition to the information provided below, further information can also be found in US 2010/0161023 to Cohen et al.

For some applications, a virtual tool is positioned upon the road map, and/or upon the stabilized images. Typically, the positioning of a virtual tool is an intermediate step leading to the selection and positioning of a corresponding actual tool. For some applications, techniques for the generation and positioning of a virtual tool described in WO 08/107,905 to Iddan, which is incorporated herein by reference, are used in combination with techniques described herein. For some applications, image tracking is applied to a stream of image frames to facilitate the positioning of a tool, deployment of a tool, the deployment of an already-deployed tool (such as by post-dilatation of a balloon within an already-deployed stent), post-deployment analysis of a deployed tool, general observations, or any combination thereof.

For some applications, image tracking is performed with respect to a radiopaque (or otherwise visible) segment or marker(s) of the tool, which is/are visible in most or all image frames and are identified automatically by means of image processing. The markers can also be staggered relative to one another, which facilitate determining its rotational orientation. Further detail regarding suitable marker configurations can be found in U.S. Provisional Application 61/740,762 to Spencer et al, incorporated by reference herein.

The tool is aligned in image frames of the image stream, based on the identified markers or segment of the tool. For example, the image frames may be aligned such that markers in at least most of the image frames are aligned. The aligned image frames are displayed as an image stream. For some applications, image frames are tracked in the aforementioned manner, but with respect to a portion of the subject's anatomy, for example, vascular calcification of the subject.

For some applications, the tool with respect to which image frames are tracked is a balloon, a marker wire, a guide wire, a stent, an atherectomy catheter, an endoluminal imaging catheter (e.g., a catheter that uses an imaging modality that is MRI, OCT, IVUS, NIRS, ultrasound, or any combination thereof), and/or an endoluminal measurement catheter (e.g., an FFR catheter).

The identification of the markers or radiopaque segments is typically performed automatically by the system. For some applications, the identification is performed within one or more entire image frames. Alternatively, the identification is performed within an ROI which was previously set by the system and/or the user. Further alternatively, the ROI is automatically set by the system to include regions in which the markers are more likely to appear, and to exclude regions in which the markers are less likely to appear. For some applications, the ROI is indicated graphically, overlaid upon the image stream.

For some applications, markers are identified by the user designating a region within the image in which the markers are more likely to appear, followed by the system automatically identifying the markers within that region. For some applications, the user is subsequently prompted to confirm the identification selection of markers by the system. In accordance with respective applications, the region is designated by the user within a dynamic image stream, or within a static image frame taken from within the image stream. Alternatively, the user clicks on (or otherwise indicates) the device or the vicinity of the device, and, in response, the image tracking with respect to the device markers or segments commences.

Once the markers have been identified in one or more image frames, then the system typically continues to identify (i.e., detect) those markers automatically in the subsequent image frames along the image stream or a segment thereof, and displays a tracked image stream. Typically, in order to detect the markers, the system accounts for phenomena such as the following:

(1) In some image frames, contrast agent may hide, or partially hide the markers. Typically, if the markers are not visible due to the contrast agent in a given frame, then that frame is skipped and is not used in the image-tracked image stream. For some applications, the system identifies markers in image frames in which the markers are partially hidden by the contrast agent, and the image frames are used in the image-tracked image stream.

(2) A fluoroscopic image is typically a two-dimensional projection of the three-dimensional portion of the subject's body that is being imaged. This may result in darkened regions, which appear similar to markers, but which are not markers. For example, regions of the image in which vessels (particularly vessels that contain contrast agent) are projected onto the two-dimensional image such that they appear to be crossing each other, may appear as a dark circle. Similarly, regions in which a tool crosses a vessel, two tools cross each other, a tool crosses the edge of a rib, or a vessel crosses the edge of a rib, may appear similar to a marker. Examples of such tools include a wire (such as a CABG wire, or a guide wire), a CABG clip, an electrode, a lead, and/or a catheter lumen.

(3) In a dynamic image stream, markers may be blurred due to the rapid movement of blood vessels.

Image tracking with respect to a portion of the tool typically effects a visual separation between two elements of the motion of the tool positioned within a vessel attached to the heart. The motion of the tool together with the vessel is typically hidden, while the motion of the tool relative to the vessel typically remains visible. For some applications, such separation of motion elements typically facilitates the ability of the user to determine the extent of the motion of the tool relative to the vessel (e.g., cyclic longitudinal motion within the blood vessel) in the course of the heart's motion cycle. That, in turn, typically enables the user to determine the importance of deploying the tool at a specific phase in the motion cycle, and if so, at which specific phase, and location.

Reference will now be made to a balloon atherectomy device used in conjunction with image tracking. Balloon atherectomy devices include one or more cutting elements that can be used to scrap atheroma deposits from the luminal surface. The following description is equally applicable to other devices, including atherectomy catheters. When placing a balloon relative to a designated lesion within a coronary artery, image tracking is performed on the radiopaque marker(s) of the balloon. Consequently, the motion of the balloon together with the artery, in the course of the heart's motion cycle, is typically hidden. At the same time, the motion of the balloon relative to the artery, in the course of the heart's motion cycle, typically remains visible. Consequently, the user can observe (typically while being demonstrated by contrast agent) the location of the balloon prior to its inflation, and/or the stent prior to its deployment, at a systolic or end-systolic phase versus a diastolic or end-diastolic phase. Based on the observed locations of the balloon or the stent, deployment of the balloon or the stent at a desired location is timed to the phase in the cardiac cycle at which the pre-deployment position is at the desired location. For some applications, techniques are provided for facilitating the determination of the location of a tool, such as a balloon, with respect to an artery, during image sequences for which a contrast agent has not been administered to the subject. For some applications, the current locations of radiopaque markers or radiopaque segments of the tool are determined with respect to a road map that was generated from a previously-acquired angiogram. Typically, the procedure includes some or all of the following phases, and is typically performed in real time:
a. A road map is generated, typically automatically, for example, according to techniques described hereinabove. Typically, the road map is updated automatically during the procedure, in response to the system detecting that a new angiographic sequence has commenced, as described hereinabove. For some applications, commencement of the new angiographic sequence is detected even when the angiographic sequence is performed under fluoro mode. For some applications, the shape of a vessel through which tools are inserted changes in the course of the procedure due to occlusions being reduced, the tool itself straightening the artery, and/or other reasons, and the road map is updated in order to account for these changes.
b. Features residing within the vessel at a relatively fixed location are identified, such features being observable even in images generated in the absence of contrast agent. Such features may include a distal portion of the guiding catheter through which the tool is inserted, a radiopaque portion of the guide wire upon which the tool is inserted, and/or other features. For some applications, the identification of such features is automatic, or semi-automatic (i.e., requiring some user interaction but less than would be required without using the techniques described herein), for example, in accordance with techniques described hereinabove. For some applications, the entire length of the guide wire (or of the catheter carrying the tool) is identified, for example using techniques similar to the ones described hereinabove for the automatic identification of center lines.
c. A current image stream of the tool inside the blood vessel is generated. The markers or radiopaque segments of the tool that is currently inserted into the blood vessel are identified in the image stream, typically automatically and typically in real time, according to techniques described hereinabove. The markers or radiopaque segments are identified even in current images generated in the absence of contrast agent (and in which the artery itself is not visible). The location of the markers with respect to the observable features is determined based upon the current image stream.
d. The tool markers or radiopaque segments are projected, typically automatically and typically in real time, upon the previously-generated road map. Typically, the current location for marker projection within the road map is calculated relative to the aforementioned observable features described in step b. For example, the current distance(s) of the markers from the observable feature(s) (as determined in step c) may be applied along the applicable vessel in the road map in order to determine the location on the road map at which the markers will be projected.

For some applications, the angiogram from which the road map is generated is gated to a specific phase in the cardiac cycle (e.g., the end-diastolic phase), and the location of the markers with respect to the observable features is determined in a current image frame that is also gated to that phase.
In an alternative application, the road map is projected (continuously or in a gated manner) upon the image stream that contains the markers or radiopaque segments (as opposed to the image stream being projected upon the road map).

The following are exemplary imaging devices that can be used in accordance with methods of the invention. As discussed above, the imaging device can be inserted into the lumen to be treated in order to obtain intraluminal data, which is then co-registered with an external imaging modality.

Exemplary imaging catheters that may be used to obtain image data for diagnosis of the stenosis and tissue characterization prior to atherectomy are shown in FIGS. 6 and 7. The catheter shown in FIG. 6 is a generalized depiction of a phased array imaging catheter. Phased array imaging catheter 900 is typically around 200 cm in total length and can be used to image a variety of vasculature, such as coronary or carotid arteries and veins. Phased array catheter 900 can be shorter, e.g., between 100 and 200 cm, or longer, e.g., between 200 and 400 cm. When the phased array imaging catheter 400 is used, it is inserted into an artery along a guidewire (not shown) to the desired location (i.e. location of the vascular access site). Typically a portion of catheter, including a distal tip 410, comprises a guidewire lumen (not shown) that mates with the guidewire, allowing the catheter to be deployed by pushing it along the guidewire to its destination. The catheter, riding along the guidewire, can obtain images surrounding the vascular access site and within the vascular access site (e.g. within the fistula or AV graft).

An imaging assembly 420 proximal to the distal tip 410, includes a set of transducers that image the tissue with ultrasound energy (e.g., 20-50 MHz range) and a set of image collectors that collect the returned energy (echo) to create an intravascular image. The array is arranged in a cylindrical pattern, allowing the imaging assembly 420 to image 360° inside a vessel. In some embodiment, the transducers producing the energy and the collectors receiving the echoes are the same elements, e.g., piezoelectric elements. Because the phased array imaging catheter 400 does not have a rotating imaging assembly 420, the phased array imaging catheter 400 does not experience non-uniform rotation distortion.

Suitable phased array imaging catheters, which may be used to assess vascular access sites and characterize biological tissue located therein, include Volcano Corporation's Eagle Eye® Platinum Catheter, Eagle Eye® Platinum Short-Tip Catheter, and Eagle Eye® Gold Catheter.

FIG. 7 is a generalized depiction of a rotational imaging catheter 500 incorporating a proximal shaft and a distal shaft of the invention. Rotational imaging catheter 500 is typically around 150 cm in total length and can be used to image a variety of vasculature, such as coronary or carotid arteries and veins. When the rotational imaging catheter 500 is used, it is inserted into an artery along a guidewire (such as a pressure/flow guidewire) to the desired location. Typically a portion of catheter, including a distal tip 510, comprises a lumen (not shown) that mates with the guidewire, allowing the catheter to be deployed by pushing it along the guidewire to its destination.

An imaging assembly 520 proximal to the distal tip 510, includes transducers that image the tissue with ultrasound energy (e.g., 20-50 MHz range) and image collectors that collect the returned energy (echo) to create an intravascular image. The imaging assembly 520 is configured to rotate and travel longitudinally within distal shaft 530 allowing the imaging assembly 520 to obtain 360° images of vasculature over the distance of travel. The imaging assembly is rotated and manipulated longitudinally by a drive cable (not shown). In some embodiments of rotational imaging catheter 500, the distal shaft 530 can be over 15 cm long, and the imaging assembly 520 can rotate and travel most of this distance, providing thousands of images along the travel. Because of this extended length of travel, the speed of the acoustic waves through distal shaft 530 should ideally be properly matched, and that the interior surface of distal shaft 530 has a low coefficient of friction. In order to make locating the distal shaft 530 easier using angioscopy, distal shaft 530 optionally has radiopaque markers 537 spaced apart at 1 cm intervals.

Rotational imaging catheter 500 additionally includes proximal shaft 540 connecting the distal shaft 530 containing the imaging assembly 520 to the ex-corporal portions of the catheter. Proximal shaft 540 may be 100 cm long or longer. The proximal shaft 540 combines longitudinal stiffness with axial flexibility, thereby allowing a user to easily feed the catheter 500 along a guidewire and around tortuous curves and branching within the vasculature. The interior surface of the proximal shaft also has a low coefficient of friction, to reduce NURD, as discussed in greater detail above. The ex-corporal portion of the proximal shaft 540 may include shaft markers that indicate the maximum insertion lengths for the brachial or femoral arteries. The ex-corporal portion of catheter 500 also include a transition shaft 550 coupled to a coupling 560 that defines the external telescope section 565. The external telescope section 565 corresponds to the pullback travel, which is on the order of 150 mm. The end of the telescope section is defined by the connector 570 which allows the catheter 500 to be interfaced to an interface module which includes electrical connections to supply the power to the transducer and to receive images from the image collector. The connector 570 also includes mechanical connections to rotate the imaging assembly 520. When used clinically, pullback of the imaging assembly is also automated with a calibrated pullback device (not shown) which operates between coupling 2560 and connector 570.

The imaging assembly 520 produces ultrasound energy and receives echoes from which real time ultrasound images of a thin section of the blood vessel are produced. The transducers in the assembly may be constructed from piezoelectric components that produce sound energy at 20-50 MHz. An image collector may comprise separate piezoelectric elements that receive the ultrasound energy that is reflected from the vasculature. Alternative embodiments of the imaging assembly 520 may use the same piezoelectric components to produce and receive the ultrasonic energy, for example, by using pulsed ultrasound. Another alternative embodiment may incorporate ultrasound absorbing materials and ultrasound lenses to increase signal to noise.

Suitable rotational IVUS catheters, which may be used to assess vascular access sites and characterize biological tissue located therein, include Volcano Corporation's Revolution® 45 MHz Catheter.

Further, IVUS technology, for phased-array and rotational catheters, is described in more detail in, for example, Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, U.S. Pub. 2009/0284332; U.S. Pub. 2009/0195514 A1; U.S. Pub. 2007/0232933; and U.S. Pub. 2005/0249391 and other references well known in the art relating to intraluminal ultrasound devices and modalities.

In addition to IVUS, other intraluminal imaging technologies may be suitable for use in methods of the invention for assessing and characterizing vascular access sites in order to diagnose a condition and determine appropriate treatment. For example, an Optical Coherence Tomography catheter may be used to obtain intraluminal images in accordance with the invention.

OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable minor, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference minor, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

In yet another embodiment, the imaging catheter for use in methods of the invention is an optical-acoustic imaging apparatus. Optical-acoustic imaging apparatus include at least one imaging element to send and receive imaging signals. In one embodiment, the imaging element includes at least one acoustic-to-optical transducer. In certain embodiments, the acoustic-to-optical transducer is an Fiber Bragg Grating within an optical fiber. In addition, the imaging elements may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interferences in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

Exemplary optical-acoustic imaging assemblies are disclosed in more detail in U.S. Pat. Nos. 6,659,957 and 7,527,594, 7,245.789, 7447,388, 7,660,492, 8,059,923 and in U.S. Patent Publication Nos. 2008/0119739, 2010/0087732 and 2012/0108943.

In certain embodiments, angiogram image data is obtained simultaneously with the intraluminal image data obtained from the imaging catheters. In such embodiments, the imaging catheter may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

According to certain aspects of the invention, the obtained image data and/or functional flow data is processed to characterize biological material at the desired site of the atherectomy. The characterization allows one to determine with specificity the severity of the atheroma and guides treatment of the stenosis. The processing step may be performed by an image processing computer 26 coupled to an imaging catheter. The imaging catheter may be directed coupled to the image processing computer or coupled to a system controller that allows for manipulation of the imaging catheter.

Referring now to FIG. 4, the imaging catheter 400, 500 may be coupled to and coordinated by a system controller 600. The system controller 600 may control the timing, duration, and amount of imaging. As shown in FIG. 4, the system controller 600 is additionally interfaced with image processing computer 26 (also shown in FIG. 1). According to certain embodiments, the processor 1065 of the image processing computer 26 performs tissue/blood characterization, thereby allowing the viewed and assessed images to be the basis for defining parameters for identifying a condition and developing a therapeutic mode for treating the condition. The system 1000 also includes a display 580 and a user interface that allow a user, e.g. a surgeon, to interact with the images (including tissue characterization) and to control the parameters of the treatment.

As shown in FIG. 4, the system controller 600 is interfaced to an image processing computer 1060 that is capable of synthesizing the images and tissue measurements into easy-to-understand images. The image processing computer 26 is also configured to analyze the spectrum of the collected data to determine tissue characteristics, a.k.a. virtual histology. As discussed in greater detail below, the image processing will deconvolve the reflected acoustic waves or interfered infrared waves to produce distance and/or tissue measurements, and those distance and tissue measurements can be used to produce an image, for example an IVUS image or an OCT image. Flow detection and tissue characterization algorithms, including motion-detection algorithms (such as CHROMAFLO (IVUS fluid flow display software; Volcano Corporation), Q-Flow, B-Flow, Delta-Phase, Doppler, Power Doppler, etc.), temporal algorithms, harmonic signal processing, can be used to differentiate blood speckle from other structural tissue, and therefore enhance images where ultrasound energy back scattered from blood causes image artifacts.

In certain embodiments, the image processing may additionally include spectral analysis, i.e., examining the energy of the returned acoustic signal at various frequencies. Spectral analysis is useful for determining the nature of the tissue and the presence of foreign objects. A plaque deposit or neointimal hyperplasia, for example, will typically have different spectral signatures than nearby vascular tissue without such plaque or neointimal hyperplasia, allowing discrimination between healthy and diseased tissue. Such signal processing may additionally include statistical processing (e.g., averaging, filtering, or the like) of the returned ultrasound signal in the time domain. The spectral analysis can also be used to determine the tissue lumen/blood border. Other signal processing techniques known in the art of tissue characterization may also be applied.

Other image processing may facilitate use of the images or identification of features of interest. For example, the border of a lumen may be highlighted or thrombus or plaque deposits may be displayed in a visually different manner (e.g., by assigning thrombus a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. In other embodiments, similar techniques are used to discern the extent and severity of the neointimal hyperplasia. Other measurements, such as flow rates or pressure may be displayed using color mapping or by displaying numerical values. In some embodiments, the open cross-sectional area of the lumen is colorized with red to represent the blood flux. Thus, by using virtual histology (spectral analysis), methods of the invention allow one to assess the type and severity of one or more conditions present within the vascular access site. In doing so, the need for treating the condition(s) and the type of treatment best suited for treating the condition may be determined.

In addition to the above disclosed systems, the following systems for detecting and characterizing plaque and biological tissue using virtual histology are disclosed in U.S. Pat. No. 6,200,268 entitled “VASCULAR PLAQUE CHARACTERIZATION” issued Mar. 13, 2001, U.S. Pat. No. 6,381,350 entitled “INTRAVASCULAR ULTRASONIC ANALYSIS USING ACTIVE CONTOUR METHOD AND SYSTEM” issued Apr. 30, 2002, U.S. Pat. No. 7,074,188 entitled “SYSTEM AND METHOD OF CHARACTERIZING VASCULAR TISSUE” issued Jul. 11, 2006, U.S. Pat. No. 7,175,597 entitled “NON-INVASIVE TISSUE CHARACTERIZATION SYSTEM AND METHOD” issued Feb. 13, 2007, U.S. Pat. No. 7,215,802 entitled “SYSTEM AND METHOD FOR VASCULAR BORDER DETECTION” issued May 8, 2007, U.S. Pat. No. 7,359,554 entitled “SYSTEM AND METHOD FOR IDENTIFYING A VASCULAR BORDER” issued Apr. 15, 2008, and U.S. Pat. No. 7,463,759 entitled “SYSTEM AND METHOD FOR VASCULAR BORDER DETECTION” issued Dec. 9, 2008.

In addition to tissue characterization, methods of the invention may also utilize functional flow measurements obtained at the vascular access site to assess the condition and determine course of treatment. Functional flow measurements allow one to determine pressure and flow differences at the vascular access site. Accordingly, imaging catheters of the invention may be equipped with one or more data collectors used to obtain functional flow measurements. Alternatively or in addition to, a guidewire with data collectors can be used alone or in combination with the imaging catheter to obtain the functional flow measurements (e.g., by using a pressure and/or flow guidewire and running the imaging catheter over that guidewire).

FIG. 5 shows a sensor tip 700 of a guidewire 401 that may be suitable to use with methods of the invention. Guidewire 401 will include one of pressure sensor 404 and ultrasound transducer 501. In general, guidewire 401 will sensor housing 403 for pressure sensor 404, ultrasound transducer 501, or both and may optionally include a radiopaque tip coil 405 distal to proximal coil 406. The radiopaque tip coil allows one to visualize the guidewire in angiograms.

Pressure sensor 404 can detect a lack of a pressure gradient, indicating that the fistula is not restrictive enough (i.e., if blood flows through the fistula too freely, it will not also flow to distal extremities of that limb of the body, leading to distal ischemia). It may be found, for example, that a AP of less than 20 or 30 mmHg is problematic. Pressure sensors and their use are described in U.S. Pub. 2009/0088650 to Corl. Ultrasound transducer 501 may include a forward-looking IVUS and can give the velocity of flow. Velocity data may be derived by the computer in the system from the Doppler frequency shifts detected in the ultrasound echo signals. Obtaining Doppler velocity is discussed in U.S. Pub. 2013/0303907 to Corl and U.S. Pub. 2007/0016034 to Donaldson. While the pressure sensor 404 and ultrasound transducer 501 are described as components of a guidewire, it is contemplated that the pressure sensor and ultrasound can transducer can also be incorporated into an imaging guidewire.

Guidewire 700 may comprise a flexible elongate element having proximal and distal ends and a diameter of 0.018″ or less as disclosed in U.S. Pat. No. 5,125,137, U.S. Pat. No. 5,163,445, U.S. Pat. No. 5,174,295, U.S. Pat. No. 5,178,159, U.S. Pat. No. 5,226,421, U.S. Pat. No. 5,240,437 and U.S. Pat. No. 6,106,476, all of which are incorporated by reference herein. Guidewire 700 can be formed of a suitable material such as stainless steel, Nitinol, polyimide, PEEK or other metallic or polymeric materials having an outside diameter for example of 0.018″ or less and having a suitable wall thickness, such as, e.g., 0.001″ to 0.002″. This flexible elongate element is conventionally called a hypotube. In one embodiment, the hypotube may have a length of 130 to 170 cm. Typically, such a guide wire may further include a stainless steel core wire extending from the proximal extremity to the distal extremity of the flexible elongate element to provide the desired torsional properties to facilitate steering of the guide wire in the vessel and to provide strength to the guidewire and prevent kinking.

In a preferred embodiment, methods of the invention employ a Doppler guidewire wire sold under the name FLOWIRE by Volcano Corporation, the pressure guidewire sold under the name PRIMEWIRE PRESTIGE by Volcano Corporation, or both.

In certain aspects of the invention, an atherectomy catheter is provided for use in practicing the methods described above. The atherectomy catheter has radiopaque markers (or otherwise visible markers) thereon that are automatically identifiable by means of image processing as described herein. Any type of atherectomy catheter may be used in accordance with the invention, including rotational atherectomy catheters, laser atherectomy catheters, directional atherectomy catheters, transluminal extraction atherectomy catheters, and orbital atherectomy catheters, each of which is described in further detail below. In further aspects of the invention, the atherectomy catheter is configured with one or more imaging sensors positioned thereon. In certain embodiments, the image sensor is an OCT optical sensor, but in other embodiments, the imaging sensor is an IVUS transducer. IVUS catheters are already known in the art. See for example, US Pub. Nos. 2011/0010925; 2010/0234736; 2010/0179426; 2010/0160788; 2009/0018393, each of which is incorporated by reference herein. Adapting these disclosures to arrive at the imaging atherectomy catheter of the present invention is within the skill of the art. Images obtained from the imaging atherectomy catheter, such as IVUS images, can be used to refresh the IVUS data in the coregistered set of information. In this manner, the IVUS-coregistered image is “refreshed” as cutting is performed.

Once the modalities have been coregistered and plaque has been identified using the coregistered data sets, the physician can then proceed with the atherectomy to remove the plaque. Atherectomy is a procedure that clears blockages in the coronary and peripheral arteries in order to improve blood flow to the heart and relieve symptoms of artery disease. An atherectomy catheter clears peripheral and coronary arteries by grinding, aspirating, or cutting away atherosclerotic plaque build-up.

There are several different devices used for atherectomy. Although atherectomy devices share some commonalties, each device has its own unique design and procedure. Methods of the invention can encompass identifying the type of plaque within the vessel and selecting the appropriate atherectomy device and procedure based on such information. Typically, the devices are threaded onto a catheter or guidewire and usually inserted through the femoral artery. The procedure uses the normal system of guidewires common to most catheterization procedures.

A brief overview of the various types of atherectomy and related devices suitable for use in practicing the invention is now provided. The following examples are not intended to be limiting as all atherectomy catheters and procedures are encompassed by the invention.

Rotational atherectomy involves inserting a small drill into the arteries to grind up plaque and increase blood flow to the heart. Rotational atherectomy is indicated for hardened, calcified plaque or when stenting would cause plaque to dislodge in non-uniform plaque formation. In rotational atherectomy, a high speed rotating metallic burr abrades calcified plaque in the arteries into millions of microscopic particulates. The particles are then removed from the bloodstream by the reticuloendithelial system in the liver, lung, and spleen. Risks associated with rotational atherectomy include bleeding around the heart, injury to the artery, tearing of the artery, and heart attack. An exemplary rotational atherectomy device suitable for practicing the invention is the Rotablator© device marketed by Boston Scientific.

Directional atherectomy is used to excise atherosclerotic plaque with an instrumented directional atherectomy catheter. The directional atherectomy catheter is equipped with a rotating cutter affixed to an elastic drive shaft, encased in a cylindrical stainless steel housing. The cutter contacts the plaque through a small window located in the housing. An inflated balloon located opposite the cutting window holds the catheter in place and pushes the plaque into the cutting window. During the procedure, the cutter begins on the proximal side of the window and is advanced across the occlusion distally, shaving away at the plaque and pushing the debris into the collecting chamber of the nose cone. Although the risk associated with directional atherectomy is relatively low, complications can include tearing of arterial walls which leads to the occlusion of the injured artery, and/or bleeding in the proximity of the heart. An exemplary directional atherectomy catheter for use in practicing the invention is the SilverHawk Catheter TM by Covidien.

The transluminal extraction catheter is an atherectomy device that can extract plaque and thrombi from diseased blood vessels simultaneously. In transluminal extraction atherectomy, the plaque is cut away from the arterial wall and the particles are subsequently extracted through the center of the catheter by vacuum suction and collected in a vacuum bottle attached to the proximal end of the device. Transluminal extraction atherectomy procedures are best suited for clearing out lesions containing both thrombi and plaque, plaque in saphenous vein grafts prior to use in bypass surgery, and blockages that occur in aged bypass grafts. Transluminal extraction atherectomy is associated with significant risk, including perforation, death, sidebranch occlusion, myocardial infarction, acute closure of the blood vessel, and distal embolization.

Orbital atherectomy is another atherectomy procedure. It is similar to rotational atherectomy in that it abrades plaque using an abrasive burr spinning at high speeds. Also like rotational atherectomy, the grit size and high rotational speed of orbital atherectomy devices makes the tissue debris small enough to pass through the circulatory system harmlessly, minimizing the potential for distal embolic complications. However, orbital devices have key differences from rotational devices, including the location of the burr on a compressible coil consisting of three helically-wound wires and the orbital path of the device around the periphery of the lumen. This orbital motion allows the bun to attack the plaque as it moves in a specific direction, in contrast to the burr of a rotational device, which remains in one place. The design of orbital atherectomy catheters enable the physician to control the diameter of plaque to be removed by varying rotational velocity. For example, a slow-moving bun will not cut deeply into plaque, leading to superficial ablation. If the rotational velocity is increased, however, the cut will be deeper. Suitable orbital atherectomy catheters for use in practicing the invention are available from Cardiovascular Systems, Inc.

Laser atherectomy is yet another atherectomy procedure used primarily in the peripheral arteries. Peripheral atherectomy uses a catheter that emits a high energy light (laser) to unblock the artery. The catheter is maneuvered through the vessel until it reaches the blockage. Laser energy is used to essentially vaporize the blockage inside the vessel, resulting in increased blood flow. Exemplary laser atherectomy devices for use in practicing the invention are available from Spectranetics.

The atherectomy may be performed with an extraction tool exemplified in FIGS. 10-13. In certain embodiments, the extraction tool includes a distal end that can be extended from a lumen of an interventional catheter. The distal end of the extraction tool includes one or more cutting elements. Typically, a proximal portion of the extraction tool is formed as part of or operably coupled to a drive shaft. The drive shaft may be coupled to a motor to provide rotational motion using any conventional means. A drive shaft suitable for use to impart rotation of the extraction tool is described in, for example, U.S. Pat. No. 5,348,017, U.S. Patent Publication No. 2011/0306995, and co-assigned pending U.S. Publication No. 2009/0018393 (as applied to rotating imaging sensors). Rotation of the drive shaft causes rotation of the distal end of the extraction tool. In operation, the distal end of the extraction tool is deployed from the tool lumen of a catheter. Forward movement and/or rotation of the distal end of the extraction tool cause the one or more cutting element to engage with the plaque or other unwanted substances within a vessel. The cutting elements shave, morcellate, grind, or cut off plaque thrombosis, or other material blocking the vascular access site from the luminal surface to clear the occlusion of the sclerotic vessel.

In certain embodiments, the extraction tool of an atherectomy catheter further defines a removal lumen extending from an opening located at the distal end of the extraction tool to an opening connected to a vacuum source. The vacuum source removes, via suction, plaque, thrombosis, or other material blocking the vascular access site that has been shaved, morcellated, or cut off from the luminal surface. Alternatively, a catheter itself may include a removal lumen that extends from the distal end of the imaging catheter to an opening operably associated with a vacuum source. In this embodiment, morcellated or shaved plaque/blood clot can be suctioned from the vessel through the removal lumen of the catheter.

The cutting elements used in the present invention will usually be formed from a metal, but could also be formed from hard plastics, ceramics, or composites of two or more materials, which can be honed or otherwise formed into the desired cutting edge. In certain embodiments, the cutting blades are formed as coaxial tubular blades with the cutting edges defined in aligned apertures therein. It will be appreciated that the present invention is not limited to any particular cutting element, and the cutting element may include a variety of other designs, such as the use of wiper blades, scissor blades or the like. The cutting elements can have razor-sharp smooth blade edges or serrated blade edges. Optionally, the cutting edge of either or both the blades may be hardened, e.g., by application of a coating. A preferred coating material is titanium nitride.

FIGS. 10-13 depict various embodiments of a distal end of the extraction tool suitable for use in methods of the invention. The extraction tool may be used alone or may be extended out of a catheter. Although not shown, the distal end of the extraction tools depicted in FIGS. 10-13 can also include one or more imaging elements or one or more functional sensors. The imaging elements and functional sensors can be used to obtain real-time data during the atherectomy procedure.

As shown in FIG. 10, the distal end 1200 of the extraction tool includes a helical cutting element 1205. The helical cutting element 1205 has a spiral-fluted shape. The edges 1260 of the spiral are sharp blades. When rotated, the helical cutting element 1205 grounds plaque within the vessel. The tip 1265 of the helical cutting element 1205 can be formed as a bladed point. The bladed point tip will assist in morcellating plaque/thrombosis that may be present in front of the extraction tool.

FIG. 11 depicts a distal end 1200 of an extraction tool according to one embodiment. The distal end 1200 of the extraction tool includes a recessed cutting element 1275. The recessed cutting element 1275 includes a recess 1260 within the distal end 1200 formed by edges 1260. One or more of the edges 1260 that form the recess 1260 constitute cutting blades. Optionally and as shown, the extraction tool includes a removal lumen 1220 and the recess 1260 provides access to the removal lumen 1220. The removal lumen 1220 can extend along the length of the extraction tool and operably couple to a vacuum source. In operation, the recessed cutting element 1275 is distally deployed from the tool lumen of the imaging catheter. The recessed cutting element 1275 can be moved forward and backwards and rotated to shave off or morcellate any plaque or unwanted substance that is placed within the recess 1260 via the blade edges 1260. The shaved off or morcellated material can be removed from the vessel through the removal lumen 1220.

FIG. 12 depicts a distal end 1200 an extraction tool according to another embodiment. The extraction tool includes a tubular member with a bladed end 1225 at the distal end 1220. The bladed end 1225 is formed by a sharp edge 1280. The bladed end 1225 can be open or closed. As shown in FIG. 12, the bladed end is open and includes opening 1285. The opening 1285 leads to a removal lumen 1220. In order to morcellate plaque and other unwanted substances, the distal end 1200 of extraction tool is deployed from the tool lumen of the imaging catheter. As the distal end 1200 is moved forward and rotated, the sharp edge 1280 cuts through and morcellates unwanted material (plaque/thrombus) present in front of the distal end 1200. The shaved off or morcellated material can be removed from the vessel through the removal lumen 1220.

FIG. 13 depicts the distal end 1200 of an extraction tool according to yet another embodiment. The extraction tool includes an outer tubular member 1210 that defines a removal lumen 1230 and an inner tubular member 1290 disposed within the removal lumen 1230. The outer tubular member 1210 includes a window 1305. The removal lumen 1230 can be operably coupled to a vacuum source. The inner tubular member 1290 can be moved forward and backward and rotated with respect to the outer tubular member 1210. The inner tubular member includes the same elements as the extraction tool shown in FIG. 12. The inner tubular member 1290 includes a bladed end 1295. The bladed end 1295 can be open or closed. The bladed end 1295 is formed by a sharp edge 1300. In operation, the distal end 1200 of the extraction tool is deployed from the tool lumen of the imaging catheter. The window 1305 of the outer tubular member 1210 is placed against plaque 1310 protruding from the vessel wall 1350. The inner tubular member 1290 can be moved forward and backwards and rotated within outer tubular member to morcellate and shave off any plaque placed within the window 1305. Removed plaque can be suctioned out of the vessel through the removal lumen 1230.

According to certain embodiments, methods of the invention further include assessing the atherectomy site after the interventional procedure. The intraluminal image data can be obtained with any one of the imaging catheters (e.g., IVUS or OCT) described above, or the intraluminal image data can be obtained from, for example, an imaging element located on the interventional catheter. The intraluminal image data is then reviewed to determine the success of the interventional therapy. In certain embodiments, the intraluminal image data of the treated atherectomy site is processed to characterize biological present after treatment. The images and characterization can then be assessed in order to identify whether a condition still exists at the treated atherectomy site, and, if a condition exists, to determine if further treatment is necessary to treat the identified condition. Any of the above therapeutic modes for performing atherectomy can be used for the further treatment. This process can be repeated until the identified condition is fully treated.

In further embodiments, methods of the invention also provide for long-term follow up assessments to continually monitor the treated vascular access site. For example, the follow-up assessments may be scheduled for 3, 6, 9, and 12 months after the intervention therapy.

A common risk in all these procedures is that the atherectomy device will cut beyond vessel plaque and damage healthy tissue. The present invention mitigates this risk by using coregistered sets of data to clearly identify plaque and then performing the atherectomy based on information provided in the coregistered data sets. Methods of the invention encompass the coregistration of, for example, an IVUS image with an angiography image as described above. After the IVUS catheter used to generate the IVUS image is withdrawn from the body, the method can further involve inserting an atherectomy catheter and coregistering the location of the atherectomy catheter to the IVUS/angiography image, as described above. Plaque is identified based upon the coregistered IVUS/angiography image, and the atherectomy catheter is used to excise the identified plaque. Identification of plaque during the atherectomy may be further enhanced through the use of virtual histology, the use of image enhancement, and the ability to track the atherectomy catheter within the lumen as described herein.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for removing plaque in a vessel, the method comprising:
obtaining a first set of image data corresponding to a blood vessel using a first imaging modality;
obtaining a second set of image data corresponding to the blood vessel using a second imaging modality;
coregistering the first and second sets of image data, thereby generating a coregistered set of image data;
identifying vessel plaque in the coregistered set of image data;
inserting a plaque-removal device into the blood vessel; and
removing plaque within the vessel based on plaque identified in the coregistered set of image data.

2. The method of claim 1, wherein the first imaging modality comprises an internal imaging modality.

3. The method of claim 2, wherein the internal imaging modality is selected from intravascular ultrasound (IVUS) or optical coherence tomography (OCT.)

4. The method of claim 3, wherein IVUS comprises virtual histology intravascular ultrasound (VH-IVUS).

5. The method of claim 1, wherein the second imaging modality comprises an external imaging modality.

6. The method of claim 5, wherein the external imaging modality comprises x-ray angiography.

7. The method of claim 5, wherein the external imaging modality comprises external ultrasound.

8. The method of claim 5, wherein the external imaging is selected from magnetic resonance imaging performed with contrast or magnetic resonance imaging performed without contrast.

9. The method of claim 5, wherein the external imaging is selected from computed tomography performed with contrast or computed tomography performed without contrast.

10. The method of claim 1, wherein the plaque-removal device is an atherectomy catheter.

11. The method of claim 10, wherein the atherectomy catheter is selected from a group consisting of an orbital, a rotational, a laser, and a directional atherectomy catheter.

12. The method of claim 10, wherein the atherectomy catheter comprises at least one marker visible to the external imaging modality.

13. The method of claim 10, further comprising orienting the atherectomy catheter in space and rotational positional within the vessel based on said marker.

14. The method of claim 12, wherein the marker comprises a radiopaque marker.

15. The method of claim 10, wherein the atherectomy catheter comprises an imaging sensor positioned thereon.

16. The method of claim 11, wherein the imaging sensor comprises an IVUS transducer.

17. The method of claim 11, further comprising obtaining a set of image data using the imaging sensor of the atherectomy catheter and updating the coregistered set of image data comprising the first and second sets of image data with the atherectomy catheter image data.

18. The method of claim 1, further comprising tracking the position of the plaque-removal device after insertion into the blood vessel.

19. The method of claim 18, wherein tracking comprises detecting the markers with an external imaging modality.

20. The method of claim 15, further comprising marking on the coregistered data set of image from the first and second imaging modalities, a region where the atherectomy catheter should remove plaque.

21. The method of claim 20, further comprising updating the marked region based on image data received from the atherectomy catheter.
